(19) United States
(12) Patent Application Publication
(10) Pub. No.: US 2007/0142306 A1
(43) Pub. Date: Jun. 21, 2007

(54) Strontium-based complexes, pharmaceutical compositions and dietetic products

(75) Inventors: Jean-Claude Maurel, Castries (FR); Claude-Alain Cudennec, Bois Guillaume (FR); Patrick Poucheret, Montpellier (FR)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignee: MEDESIS PHARMA SA, Paris (FR)

(21) Appl. No.: 10/578,877

(22) PCT Filed: Nov. 15, 2004
(86) PCT No.: PCT/FR04/02912

§ 371 Date: May 11, 2006

(30) Foreign Application Priority Data
Nov. 14, 2003 (FR) 0313357

(51) Int. Cl. A61K 31/70; A61K 31/56; A61K 31/22

(52) U.S. Cl. 514/25; 514/171; 514/547

(57) ABSTRACT

The invention relates to organic complexes based on sitosterols, acylglycerols and strontium and to the use thereof in the pharmaceutical and dietetic fields. The invention also relates to methods for treating different diseases, particularly bone or blood diseases, by the administration of the aforementioned complexes. The invention further relates to pharmaceutical compositions containing said complexes, in particular for the treatment of bone diseases, such as osteoporosis, and for the treatment of blood diseases.

The invention relates to organic complexes based on sitosterols, acylglycerols and strontium and to the use thereof in the pharmaceutical field and dietetic industry. The invention also relates to methods for treating different diseases, particularly bone or blood diseases, by the administration of the aforementioned complexes. The invention further relates to pharmaceutical compositions containing said complexes, in particular for the treatment of bone diseases, such as osteoporosis, and for the treatment of blood diseases.

Acylglycerols, more particularly fatty acid acylglycerols, are present in most plants and are major constituents of plant and animal fats. Only the number of fatty acids, their position on the glycerol moiety, their chain length and the number of any unsaturated bonds they contain change from one acylglycerol to another. In particular, the acylglycerols can be mono-, di- or triacylglycerols. Sitosterol, which has two active isomers, γ and β sitosterol, is also a component of all plants. Some whole plant extracts which, like most plants, contain flavonoids, tannins, saponins, coumarins, alkaloids, triterpenes, sterols, carbohydrates and/or glycosides among their numerous components, have been described as having therapeutic activity, particularly hypoglycemic activity, such as acacia extract (Egypt. J. Pharm. Sci., 1992, 33 (1-2), 327-340), or teucrium oliverianum extract (Fitoterapia, 1984, 55(4), 227-230), for example. No link between activity and the sterol-containing fraction has been established. The work carried out by the inventors has already led to different patents describing different combinations, particularly in the form of organometallic complexes, that can be used for the treatment and/or prevention of hyperglycemia, diabetes and related diseases. A particular example is the patent application WO 96/23811 which describes organometallic complexes based on sitosterols and acylglycerols, the metal being vanadium in particular.

Osteoporosis is a disease characterized by low bone mass and deterioration of bone tissue, leading to bone fragility and its resultant increased risk of bone fractures. Osteoporosis can affect the entire skeleton and causes fractures mainly of the hip, wrist, and dorsolumbar spine. Osteoporosis is a major public health problem which is exacerbated by the ageing of the population. Furthermore, while osteoporosis often strikes people over 50 years of age, it can occur at any age. Certain factors, called risk factors, are correlated with the development of osteoporosis or increase the probability of developing the disease. For instance, it has been established that a woman is more likely to be afflicted with this disease than a man. The same is true for the elderly since bone becomes less dense and therefore more fragile with age, and for thin and/or short people, etc. In adults, the bone reserve is the result of an equilibrium between bone resorption by osteoclasts and bone formation by osteoblasts up to the age of 30 years, after which bone mineral density starts to decline. At menopause, bone loss in women accelerates due to a deficiency of estrogen which increases osteoclast activity, leading to a situation where bone resorption is greater than bone formation. Additional risk factors include amenorrhea, low estrogen levels, low testosterone levels in men, anorexia, certain drugs (such as glucocorticoids, some anticonvulsants), a diet low in calcium and vitamin D, smoking and excessive alcohol consumption.

Some preventive or curative osteoporosis treatments have been described or are currently in use, such as biphosphonates, calcitonin, hormone therapies (estrogen and progestin), selective estrogen receptor modulators (raloxifen).

A new medicament in the process of registration in the treatment of osteoporosis uses strontium as active substance. Thus, some publications, particularly Reginster J. Y., Current Pharmaceutical Design, 2002, 8, 1907-1916, describe the activity of strontium ranelate in the treatment of osteoporosis. A two-year, double-blind study in postmenopausal women who took 2 mg of strontium ranelate per day showed a significant increase in bone mineral density in the lumbar spine. The study also found that bone alkaline phosphatase (a marker of bone formation) significantly increased and that a marker of bone resorption decreased, which indicates that strontium ranelate acts on two biological activities, resulting in an increase in bone mineral density.

Studies carried out with strontium chloride and strontium ranelate have demonstrated a dual action on the cellular effectors of bone equilibrium: stimulation of osteoblast proliferation, and inhibition of osteoclast multiplication.

Therapeutic activity in animal studies required high doses of strontium (200 mg of strontium ranelate per day, corresponding to 68.2 mg of strontium metal) and was accompanied by a fairly high incorporation of strontium in bone, with an elimination turnover that caused toxic effects (osteomalacic bone lysis) at ranelate doses above 800 mg per day (four times the active dose). This deleterious effect of strontium uptake in bone is not considered toxic below these doses because it does not alter the mechanical properties of bone.

Nonetheless, according to some authors, it appears that strontium can have harmful effects if when its incorporation in bone becomes too high, particularly in the presence of concomitant renal impairment (Martine Cohen-Solal, Nephrol Dial Transplant (2002) 17 [Suppl. 2]: 30-34).

Recent studies have found a correlation between the number of osteoblasts and the number of hematopoietic stem cells. The processes inducing an increase in the number of osteoblasts induce a concomitant increase in hematopoietic stem cells (“Osteoblastic cells regulate the haematopoietic stem cell niche”, Calvi L. M. et al., Nature, Oct. 23, 2003; 425 (6960): 841-6. and “Identification of the haematopoietic stem cell niche and control of the niche size”, Zhang J. et al., Nature, Oct. 23, 2003; 425 (6960): 836-41).

Stimulation of osteoblast multiplication by strontium may therefore induce a stimulation of the hematopoietic stem cell niche.

The availability of a pharmaceutical active substance that can be used at low doses, all while displaying a pharmacological activity at least equivalent to that of currently used strontium salts, would therefore be of major interest, therapeutically (reduction of deleterious effects) and economically.

The invention therefore proposes novel organic complexes based on sitosterols, acylglycerols and strontium allowing to increase the bioavailability of strontium. As a matter of fact, the inventors have found that by administering particular organic complexes containing strontium to an animal, bone growth is observed, even at low strontium doses, all with less strontium incorporation in bone.

Therefore, according to a first aspect, the invention has as object novel organometallic complexes that can be obtained by reacting:

at least one strontium cation,

sitosterol or a plant extract containing same,

at least one mono-, one di- or one triglyceride corresponding to formula (I): 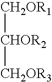
in which:
R1 is an acyl moiety of a C14 to C24 fatty acid, saturated or not, linear or branched, a hydrogen atom, or a mono-, di- or tri- galactose or glucose,
R2 is an acyl moiety of a C2 to C 18 fatty acid, linear or branched, saturated or not,
R3 is an acyl moiety of a C14 to C24 fatty acid, saturated or not, linear or branched, or a hydrogen atom.

According to a preferred variant, at least one of the groups R1 or R3 corresponding to formula (I) hereinabove is composed of an acyl moiety of oleic acid (C 18:1[cis]-9). According to another preferred variant, R2 has an unsaturated bond, preferably it is an acyl moiety of a C18 fatty acid, advantageously it represents a moiety of oleic acid or one of its double bond positional isomers (cis-6, 7, 9, 11, 12 and 13) or one of its geometric isomers. According to another variant, R2 represents an acetyl group. According to a particularly advantageous variant of the invention, R3 corresponding to aforementioned formula (I) represents the hydrogen atom.

The complexes of the invention form quite easily by mixing the three types of components while generally maintaining them at a temperature comprised between 30° C. and 40° C. for about 12 hours; the catalytic reaction of complex formation is carried out more slowly at room temperature (5 to 8 days at 25° C.); little or no complex forms if the mixture is kept in the cold.

In this manner, then, the applicant has clearly demonstrated the favorable effect of elevating the temperature, in particular between 30 and 40° C., in the reaction occurring between the components of the inventive complex.

The three types of components react very easily by dissolution in an organic solvent, for example dichloromethane, ether, chloroform, methanol, ethanol or ethyl acetate, which are then evaporated.

The strontium cation used to prepare the inventive complexes is therefore a divalent cation capable of forming a complex with the two aforementioned types of organic derivatives. Particular examples of strontium salts that can be used in the invention to supply the strontium cation are the dihalogenides, more specifically the dichloride, sulfates, hydrates, which can advantageously be dissolved in water or sometimes in alcohols. Organic strontium derivatives such as acetylacetonates, alcoholates, in particular strontium ranelate, or strontium complexes with organic solvents, for example ethers, THF, DMF, can also be mentioned. Said organic strontium derivatives are generally soluble in organic solvents, more particularly in chlorinated solvents like chloroform or dichloromethane.

Sitosterol which is used for preparing the inventive complexes can be in the β or γ form. A mixture of the two sterols can also be used.

Sitosterol is commercially available. However, it is generally in the form of a mixture with campesterol. In these commercial products generally extracted from soybean, β-sitosterol accounts for only 50% of the product, the main impurity being campesterol. Another interesting commercial source is obtained from tall-oil, in which sitosterol comprises more than 75% of the product. It is possible to obtain β-sitosterol with a purity of more than 95%, or even 99%, by proceeding as follows: the commercial mixture is recrystallized several times in acetone, which prepurifies β-sitosterol by eliminating the campestanol and sitostanol present in the mixture. Next, the prepurified compound is subjected to one to three purification steps by high pressure liquid chromatography on a preparative C18 column using a mixture of mobile phases, such as methanol, in particular 100% methanol or mixtures of methanol and acetonitrile, in particular 80:20 mixtures or any other intermediate mixture yielding sitosterol with more than 95 or even 99% purity. Purity is determined by gas chromatography.

Sitosterol can also be prepared by extraction from plants according to methods described in the literature, for example page 95 of the thesis presented by Claude Cerdon in Montpellier entitled: “Modulation of steroidal sapogenin production in response to inhibition of sterol synthesis”.

Said extraction is advantageously carried out by metal complexation according to the method described in particular in French patent 2 316 247 which provides a method for isolating 3-hydroxysteroids and 3-oxosterodes from a mixture containing said compounds.

To carry out said extraction, any plant or plant product known to contain relatively high amounts of sitosterol can be used.

Particular examples of plants or plant products containing relatively high levels of free sitosterol include olive oil, soybean oil, cotton leaves, coffee leaves, wheat germ.

It should be noted, however, that the free sitosterol fraction contains a variable proportion of the 24R and 24S isomers, depending on the plant used, said proportion not being known because there are few if any studies, and which seems to explain a better relative activity of the sterol fraction of certain plants and especially the excess of sitosterol necessary for the manufacture of novel products.

The acylglycerols corresponding to formula (I) used for the preparation of the inventive complexes can be isolated from most plants.

More generally, the chemical fraction containing said products can be extracted from labiate plants, nettle (urtica dioica and urens), sage, bugles, lucerne or alfalfa (medicago sativa), eucalyptus (globulus, delegatensis), angelica archangelica and angelica sinensis, umbelliferae, gymnema sylvestre (asclepiadaceae), marsdenia condurango, momordica charantia, gingko biloba, thistle, green tea, black tea (camelia sinensis), rhubarb, dioscorea dumetorum (dioscoreaceae), indigofera arrecta (papilionaceae), pittosporaceae, agrimonia eupatoria, curcuma xanthorrhiza (roxb.), uncaria gambier (roxb.), swertia chirayita (roxb.), resedaceae (reseda, phyteuma, lutea, alba, luteola), harpagophytum, rubiaceae, gentianaceae, asparagus racemosus, dioscorea durnetorum (dioscoraceae), hawthorn (crataegus oxyacantha), mistletoe (viscum album), mangrove (rhizophoraceae), palms, persimmon, oak, gall oak (fagaceae), bramble, hamamelis (hamamelidaceae), ratanhia (krameriaceae), purple loosestrife (lythraceae), calophyllum (clusiaceae), acacia, catechu acacia (mimosoid legumes), quebracho (anacardiaceae), grapes (vitis vinifera, ampelidaceae), blackcurrant (saxifragaceae including ribes nigrum), blueberries (erieaceae), blackberries (rubus fructicosus), elder tree, red cabbage, garlic (allium sativum), coriander (coriandrum sativum), juniper (juniperus communis), pine (abietaceae), maritime pine, cypress (cupressaceae), hibiscus, rhus (anacardiaceae), dicotyledons, ferns, gymnosperms, melianthus, rosaceae, roses, eriobotrya japonica (rosaceae), boussingaultia baselloides, malva verticillata (malvaceae), strawberry plant, citrus (rutaceae), benoite, blighia sapida (sapindaceae), hawthorn, chestnut trees (fragaceae), sumacs (anacardiaceae), myrabolans (combretaceae), bistorte, cesalpinia legumes (dividivi, tara, algarobilla), papilionoid legumes (derris, lonchcarpus, mundelea, tephrosia), lespedeza, sophora, polygonaceae, legumes, buckwheat.

In a particularly advantageous manner, unsaturated plant oils are used as source of acylglycerols, in particular first cold pressed olive oil.

As a general rule, an oil or an oil fraction having a high oleic acid content is selected as source of acylglycerols to be used in the invention, said oil generally containing a high proportion of acylglycerols that can be used according to the invention.

Examples of such oils are listed below:
olive oil having an oleic acid content (C18:1) comprised between 60 and 80%, European oils being richer in C18:1 than oils from North Africa,
sunflower oil of the so-called high oleic sunflower hybrid variety, containing 83% of C18:1 instead of 16% in regular sunflower oil,
oleic safflower oil, containing from 73 to 80% of C18:1 instead of 10 to 20% in the linoleic variety,
almond oil containing 64 to 82% of C18:1,
hazelnut oil containing 66 to 83% of C 18:1,
avocado oil the C18:1 content of which ranges from 36 to 80%.

The acylglycerol-containing fraction that can be used to prepare the inventive complexes can advantageously be prepared from olive oil in the following manner: the olive oil is prepurified by passing it through a short silica column (10 to 15 cm) under vacuum and eluting it with an organic solvent such as dichloromethane or a mixture of cyclohexane and ethyl acetate in proportions of 96:4 or any other eluent with similar polarity, so as to isolate the triglycerides present in the oil including that having a C18 chain on the carbon in position 2 of the glycerol. The silica is then washed with ethyl acetate to recover monoglycerides and diglycerides of interest in the invention, that is to say, those containing a C18 fatty acid on the carbon in position 2 of the glycerol moiety.

The fraction so obtained is then passed through a silica column and eluted with different gradients of an ethyl acetate/cyclohexane mixture comprised between 10:90 and 100:0 so as to prepare different chemical families from the oil and recover the family of interest.

According to a particular aspect of the invention, the acylglycerols used in the invention are selected in the group consisting of 1,2-diolein and 1-oleoyl-2-acetylglycerol.

The acylglycerols of the invention are also commercially available. In particular, 1-oleoyl-2-acetylglycerol and 1,2-dioleoylglycerol are commercially available at high purity (more specifically, glycerol monooleate contains approximately 44% of dioleic glycerides, among which 1,2-diolein accounts for about 14%). Such a compound is pharmaceutically acceptable (European Pharmacopeia (4^{th} Edition), USP 25/NF20, and Japanese Standard of Food Additives) and is commercialized in particular by the company Gattefossé under the name PECEOL®.

As indicated earlier, the complexes according to the invention are readily formed by simply mixing the three types of components described hereinabove. Advantageously, said mixture is prepared in an organic solvent such as dichloromethane, ether, chloroform, ethyl acetate, ethanol, and the mixture is then maintained at a temperature comprised between 30° C. and 40° C. for about 12 hours, and for a shorter time when the mixture is stirred.

In an advantageous manner, equimolar proportions of the two lipid components are used. However, these conditions are not critical, and one may advantageously use an excess of sitosterol relative to the acylglycerol in proportions of 1 to 50 depending on the nature of the sterol (24 R or 24 S).

The metal (strontium) can be used at very low doses in relation to the other two components, in particular in a ratio of 1/10 to 1/100 expressed in moles relative to acylglycerol.

The different components are formally identified by suitable analytical methods
for sitosterol: gas chromatography,
for the acylglycerol: HPLC with light diffusion detector, on a Kromasil C18 column in the presence of a mobile phase composed of isocratic acetonitrile for example.
   Gas chromatography can also be used for identification of monoglycerides.

The mass peak of the complex is generally not detectable with the usual methods, such as chemical ionization and electron impact, which can be explained by the fact that the complexes formed by these two components with the metal are usually unstable, like the majority of organometallic complexes displaying biological activity.

According to another aspect, the invention relates to the use of the aforementioned complexes as transporters of the strontium cation, the ligands bound to strontium serving to increase the bioavailability of said strontium.

Such an application is all the more important given that, generally, those skilled in the art know that the difficulty of using strontium therapeutically may be related to its toxicity above the active doses.

The organometallic strontium complexes, described in the invention, provide a bone growth activity at least equivalent to that of stontium dichloride at strontium doses (expressed as metal) that are 10, even 100 to 500 times lower than that used in the case of strontium dichloride. Furthermore, the pharmacological activity is obtained with less incorporation of strontium in bone. Thus it can be confirmed that at these doses, strontium in the form of the complex is devoid of toxicity.

The organometallic complexes described in the invention thereby optimize the bioavailability of the transported strontium, allowing the therapeutic use thereof with greater efficacy and little or no toxicity, which constitutes a considerable advantage over the state of the art.

According to another aspect, the invention also relates to pharmaceutical compositions containing at least one complex such as defined hereinabove and a pharmaceutically acceptable excipient, vehicle or support.

As pharmaceutically acceptable excipient, vehicle or support, one can use any excipient, vehicle or support known to those skilled in the art. Non-limiting examples include lactose, corn starch, glucose, gum arabic, stearic acid or magnesium stearate, dextrin, mannitol, talc, natural oils rich in essential unsaturated fatty acids and in sterol. In particular, if this proves necessary, other additives known to those skilled in the art can also be used, such as stabilizers, dessicants, binders, pH buffers.

The inventive compositions can be administered in different ways, in particular by the intramuscular, subcutaneous, sublingual, per os, transmucosal, transdermal route (by administering the composition in the form of a patch or gel).

According to another aspect, the invention relates to the use of the inventive complexes for preparing a medicament intended to be used as a regulator or stimulant of bone growth, particularly in the treatment or prevention of deficiencies or dysfunctions of bone growth, specifically in the treatment or prevention of osteoporosis.

According to another aspect, the invention relates to the use of the inventive complexes for preparing a medicament intended to stimulate the production of hematopoietic stem cells, particularly in the treatment of blood diseases involving a hematopoietic deficiency, more specifically as an adjunct to anticancer chemotherapies.

The invention further relates to a method of treatment of the aforementioned pathologies or disorders, comprising administering to a subject, particularly human, an effective dose of a complex or a pharmaceutical composition such as defined hereinabove.

The dosages and regimens by which the complex is administered vary according to the formulation, method of administration, conditions and particular characteristics of the subject to be treated. By way of comparison, strontium ranelate can be administered to an animal at doses ranging from approximately 200 to 1800 mg/kg/day. In humans, the amount of strontium ranelate necessary to obtain therapeutic activity is 2 g/day. When the strontium is bound to the complex described in the invention, the amount needed to obtain therapeutic activity is 10 to 100-fold lower, due to the higher bioavailability of the metal.

In this regard, the terms “treatment” or “treat” include both curative and prophylactic treatments. The inventive complex can be used at an early stage of the disease, or before the onset of the first symptoms, or else at an advanced stage of the disease.

In all of said uses, use is made of the fact that the bioavailability of strontium itself known to stimulate bone growth is increased by virtue of its complexation.

In all cases, complexation leads to an enhancement of the biocatalytic potency of strontium, which makes it possible to achieve activity with strontium doses that are considerably lower than those normally used.

According to another particularly important aspect, the invention also relates to dietetic products, particularly dietetic products that can be used as dietary supplements with calcium metabolism regulating activity and/or protective activity against deterioration of bone, particularly bone density and/or quality, incorporating the aforementioned complexes, as well as a method for preparing said products.

In fact, considering the method of preparation of the aforementioned complexes, they can be easily formed by adding sitosterol or a plant extract cotnaining at least one of these two forms of sitosterol and a metal salt such as defined hereinabove, to an oil rich in oleic acids (C18:1).

As indicated earlier, plant oils vary considerably in their C18:1 oleic acid contents according to the type of plant and the geographical origin of same.

For the preparation of dietetic products according to the invention, one advantageously uses a plant oil in which oleic acids represent at least 60% of the fatty acids.

According to a preferred variant, olive oil is used, preferably first cold pressed oil.

Sitosterol is advantageously added in the form of a plant extract. In particular, a commercial plant extract obtained from soybean or tall-oil can be used.

The dietetic product is obtained by simply mixing the oil, the sitosterol-rich plant extract and the strontium cation, then heating and optionally stirring the mixture.

Examples of such dietetic products according to the invention include mixtures composed of:
100 ml of oil wherein at least 60% of the fatty acids are oleic acids, in particular olive oil,
20 g of sitosterol extracted from tall-oil,
2 to 8 g of strontium salt expressed as mass of metal

Said product administered daily at a dose of 5 mL per os permits a better regulation of calcium metabolism and prevents deterioration of bone (density and/or quality). Said dietetic treatment is particularly indicated in the case of postmenopausal women for the prevention of bone and vertebral fractures.

The following examples are given solely for purposes of illustration of the invention.

### EXAMPLES

### Example 1

### Preparation of the Complex According to the Invention

1.70 g of commercial sitosterol dissolved in 10 ml of ethanol.

100 mg of a commercial mixture of 1,2-diolein and 1,3 diolein.

840 mg of strontium in the form of sulfate dissolved in 5 ml of purified water.

70 ml of soybean oil.

The mixture was heated for 10 minutes with stirring. The ethanol was then evaporated.

### Example 2

### Preparation of the Complex According to the Invention

1.70 g of commercial sitosterol dissolved in 10 ml of ethanol.

840 mg of strontium in the form of sulfate dissolved in 5 ml of purified water.

2 ml of a purified extract of mono and diglycerides extracted from palm-palm nut oil (70 to 80% oleic acid).

The mixture was heated at 35° C. for 30 minutes with stirring. The ethanol was then evaporated.

### Example 3

### Preparation of a Pharmaceutical Composition According to the Invention

A pharmaceutical composition was prepared from the complex formed in example 2. The product was placed inside gastroresistant capsules which were then administered.

### Example 4

### Preparation of a Pharmaceutical Composition According to the Invention

A pharmaceutical composition was prepared from the complex formed in example 1. The product is a liquid emulsion, ready for per os administration in humans, or by the intraperitoneal route or rectal route in animals.

### Example 5

### Preparation of an Oro-Dispersible Pharmaceutical Composition According to the Invention

A pharmaceutical composition was prepared from the complex formed in example 2 by addition to the mixture of excipients adapted to this pharmaceutical form.

### Example 6

### Pharmacological Tests

The complex according to example 4 was evaluated as follows:

Administration to female Wistar rats weighing 200 g.

Number of animals: 30.

Number of animals per group: 6 and 8.

| | | |
|---|---|---|
| Identification | Group 1 = Control (oily emulsion) | C (n = 6) |
| of groups: | Group 2 = Strontium chloride control | SrCl (n = 8) |
| | Group 3 = Treated NP05 (low dose) | NP05-L (n = 8) |
| | Group 4 = Treated NP05 (high dose) | NP05-H (n = 8) |

Treatment

Text complex: NP05 (composition produced according to the invention, in which only the quantity of strontium varies).

Active substance: Strontium.

Preparation: As per example 4

Strontium metal
(mg/kg/d)

| | | |
|---|---|---|
| Assay of active substance: | C | 0 |
| | SrCl | 68.2 |
| | NP05-H | 68.2 |
| | NP05-L | 12.15 |

Volume of administration: 2 ml/kg.

Route of administration: Rectal route.

Dose frequency: Once a day—every 24 hours.

Treatment duration: 21 days.

Tissue samples

The animals were sacrificed and the femoral bones were removed, cleaned of their tendinous and muscular attachments, then frozen.

Strontium was then assayed in the bones by atomic absorption spectrometry.

The femoral epiphyseal diameter was measured.

The assay of strontium in bone was carried out.

The results are presented in Tables 1 and 2 hereinafter.

TABLE 1
Diameter of femoral head (millimeters)

| | | |
|---|---|---|
| Group | Femur 1 | Femur 2 |

Controls

| | | |
|---|---|---|
| C 1 | 4 | 4 |
| C 2 | 3.7 | 3.7 |
| C 3 | 4.2 | 4.2 |
| C 4 | 4.2 | 4.2 |
| C 5 | 4 | 4 |
| C 6 | 3.9 | 4 |

| | |
|---|---|
| Mean | 4.01 |
| Standard deviation | 0.05 |

Strontium chloride (68.2 mg/kg)

| | | |
|---|---|---|
| Cl 1 | 4 | 4 |
| Cl 2 | 4 | 4 |
| Cl 3 | 4 | 4 |
| Cl 4 | 4 | 4 |
| Cl 5 | 4 | 4 |
| Cl 6 | 4 | 4 |
| Cl 7 | 4 | 4 |
| Cl 8 | 4 | 4.1 |

| | |
|---|---|
| Mean | 4.01 |
| Standard deviation | 0.01 |

NP 05 low dose (12.5 mg/kg)

| | | |
|---|---|---|
| L 1 | 4.3 | 4.3 |
| L 2 | 4.3 | 4.3 |
| L 3 | 4 | 4 |
| L 4 | 4.4 | 4.4 |
| L 5 | 4.1 | 4.1 |
| L 6 | 4.2 | 4.2 |
| L 7 | 4 | 4 |
| L 8 | 4.4 | 4.4 |

| | |
|---|---|
| Mean | 4.18 |
| Standard deviation | 0.04 |

NP 05 high dose (68.2 mg/kg)

| | | |
|---|---|---|
| H 1 | 4.3 | 4.3 |
| H 2 | 3.9 | 4 |
| H 3 | 4.3 | 4.3 |
| H 4 | 4.4 | 4.4 |
| H 5 | 4 | 4 |
| H 6 | 4.4 | 4.4 |
| H 7 | 4.3 | 4.2 |
| H 8 | 4.2 | 4.1 |

| | |
|---|---|
| Mean | 4.25 |
| Standard deviation | 0.04 |

| | | | | |
|---|---|---|---|---|
| Stats | C | Cl | L | H |
| C | — | NS | S | S |
| Cl | 0.968 | — | HS | HS |
| L | 0.00468 | 0.000104 | — | NS |
| H | 0.00429 | 0.000139 | 0.914 | — |

Student's test
C not different from Cl
L not different from H
L and H different from C and Cl

TABLE 2

| | |
|---|---|
| ASSAY OF CALCIUM | ASSAY OF STRONTIUM |

| | Ca concentration | | Sr concentration |
|---|---|---|---|
| Sample | (mg/g) | Sample | (mg/g) |
| C1 | 72.698 | C1 | 100.14 |
| C2 | 61.893 | C2 | 72.83 |
| C3 | 78.219 | C3 | 86.60 |
| C4 | 68.615 | C4 | 72.49 |
| C5 | 63.916 | C5 | 67.84 |
| C6 | 61.893 | C6 | 70.57 |
| Mean | 67.873 | Mean | 78.41 |
| Standard | 6.604 | Standard | 12.48 |
| deviation | | deviation | |
| Cl1 | 77.882 | Cl1 | 308.32 |
| Cl2 | 79.211 | Cl2 | 350.27 |
| Cl3 | 72.946 | Cl3 | 306.08 |
| Cl4 | 57.229 | Cl4 | 342.23 |
| Cl5 | 64.538 | Cl5 | 250.72 |
| Cl6 | 71.630 | Cl6 | 241.67 |
| Cl7 | 70.192 | Cl7 | 437.51 |
| Cl8 | 71.221 | Cl8 | 441.82 |
| Mean | 70.606 | Mean | 334.83 |
| Standard | 7.069 | Standard | 75.18 |
| deviation | | deviation | |
| L1 | 84.180 | L1 | 176.64 |
| L2 | 78.618 | L2 | 120.78 |
| L3 | 66.324 | L3 | 126.75 |
| L4 | 62.572 | L4 | 102.72 |
| L5 | 64.209 | L5 | 99.93 |
| L6 | 73.896 | L6 | 87.04 |
| L7 | 60.142 | L7 | 109.39 |
| L8 | 61.336 | L8 | 119.51 |
| Mean | 68.910 | Mean | 117.84 |
| Standard | 8.908 | Standard | 27.04 |
| deviation | | deviation | |
| H1 | 69.516 | H1 | 148.20 |
| H2 | 70.165 | H2 | 194.70 |
| H3 | 72.865 | H3 | 123.21 |
| H4 | 53.810 | H4 | 85.91 |
| H5 | 73.689 | H5 | 190.64 |
| H6 | 56.494 | H6 | 166.95 |
| H7 | 72.791 | H7 | 150.51 |
| H8 | 59.073 | H8 | 141.94 |
| Mean | 66.050 | Mean | 150.26 |
| Standard | 8.185 | Standard | 35.52 |
| deviation | | deviation | |

Comments and Results:

The duration of treatment in this study was 3 weeks, and the strontium chloride treatment group showed no effect on bone growth, even though strontium incorporation in bone was relatively high compared with the controls (×4); whereas studies in animals (Optimizing bone metabolism in osteoporosis: insight into the pharmacologic profile of strontium ranelate; Marie P J, Osteoporos Int Mar. 14, 2003 Suppl 3: 9-12) showed in a rat model that bone growth was stimulated by administration of a minimum dose of 68.2 mg of strontium (metal) in the form of strontium chloride or ranelate for 8 weeks.

On the other hand, the animals treated for only 3 weeks with strontium prepared according to the invention displayed a highly significant increase in bone growth compared with control animals or with the strontium chloride group. In two groups which received the inventive complex, activity was achieved with less strontium incorporation in bone (two-fold less for the group with the same strontium dose as Sr dichloride and 1.5-fold less for the group receiving six times less strontium).

1. Organometallic complexes produced by the process comprising reacting:
at least one strontium cation,
sitosterol or a plant extract containing same,
at least one mono-, one di- or one triglyceride corresponding to formula (I): 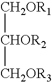
in which:
R₁ is an acyl moiety of a C14 to C24 fatty acid, saturated or not, linear or branched, a hydrogen atom, or a mono-, di- or tri-galactose or glucose,
R₂ is an acyl moiety of a C2 to C 18 fatty acid, linear or branched, saturated or not,
R₃ is an acyl moiety of a C14 to C24 fatty acid, saturated or not, linear or branched, or a hydrogen atom.

2. Complexes according to claim 1, wherein at least one of the groups R₁ or R₃ in formula (I) hereinabove is includes an acyl moiety of oleic acid.

3. Complexes according to either one of claims 1 or 2, in which R₂ has an unsaturated bond.

4. Complexes according to either one of claims 1 or 2, in which R₂ represents an acetyl group.

5. Complexes according to claim 1, in which R₃ in formula (I) represents the hydrogen atom.

6. Complexes according to claim 1, in which the diglyceride is obtained by isolation from olive oil or an oil rich in oleic acid, or an oil fraction rich in oleic acid.

7. Complexes according to claim 1, in which the strontium cation is a dihalogenide, an organic strontium derivative or a complex of strontium with organic solvents.

8. A pharmaceutical composition comprising at least one complex defined in claim 1 and a pharmaceutically acceptable vehicle, excipient or support.

9. A method for preparing a medicament intended for use as a regulator or stimulant of bone growth comprising combining the complexes of claim 1 with a pharmaceutically acceptable vehicle, excipient or support.

10. A method for preparing a medicament intended to stimulate the production of hematopoietic stem cells, comprising combining the complexes of claim 1 with a pharmaceutically acceptable vehicle, excipient or support.

11. Dietetic products comprising at least one complex defined in claim 1.

12. (canceled)

13. Complexes according to claim 3, wherein the unsaturated bond represents an oleic acid or one of the double bond positional isomers cis-6, 7, 9, 11, 12 or 13, or one of the geometric isomers thereof.

14. Complexes according to claim 7, wherein the dihalogenide is a strontium dichloride, sulfate or hydrate.

15. Complexes according to claim 7, wherein the organic strontium derivative is an acetylacetonate or an alcoholate.

16. Complexes according to claim 15, wherein the organic strontium derivative is strontium ranelate.

17. A method of treating or preventing deficiencies or dysfunctions of bone growth comprising administering complexes of claim 1 to a subject.

18. The method of claim 17, wherein said deficiency or dysfunction of bone growth is osteoporosis.

19. A method of treating blood diseases involving a hematopoietic deficit comprising administering the complexes of claim 1 to a subject.

20. The method of claim 19, wherein complexes of claim 1 are used as an adjunct to anticancer therapies.

21. A composition comprising:
at least one strontium cation,
sitosterol or a plant extract containing same,
at least one mono-, one di- or one triglyceride corresponding to formula (I): 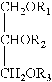
in which:
R₁ is an acyl moiety of a C14 to C24 fatty acid, saturated or not, linear or branched, a hydrogen atom, or a mono-, di- or tri-galactose or glucose,
R₂ is an acyl moiety of a C2 to C 18 fatty acid, linear or branched, saturated or not,
R₃ is an acyl moiety of a C14 to C24 fatty acid, saturated or not, linear or branched, or a hydrogen atom.
